(19) United States
(12) Patent Application Publication
(10) Pub. No.: US 2019/0076612 A1
(43) Pub. Date: Mar. 14, 2019

(54) NASAL INHALER FOR USE WITH NEBULIZER SYSTEM

(71) Applicants: Rosemary Marie Amaral, Petaluma, CA (US); James Marco Steele, Santa Rosa, CA (US)

(72) Inventors: Rosemary Marie Amaral, Petaluma, CA (US); James Marco Steele, Santa Rosa, CA (US)

(21) Appl. No.: 15/702,672

(22) Filed: Sep. 12, 2017

(51) Int. Cl. A61M 16/06; A61M 16/10; A61M 16/08

(52) U.S. Cl. CPC A61M 16/0666; A61M 39/10; A61M 16/0833; A61M 16/10

(57) ABSTRACT

A nasal inhaler for use in a nebulizer system for prolonged and continuous administration of medications. The inhaler includes an interface tube configured for connection to a nebulizer chamber, an outlet tube, and a patient interface assembly. The inhaler interface includes a hollow interface coupler having a connector tube for coupling to the interface tube, a hollow removable concentrator connected to the interface coupler and having two concentrator outlet tubes, and two nares elements, one each removably connected to one of the concentrator outlet tubes. The interface coupler and the concentrator form a plenum through which medicated air under positive pressure from the nebulizer travels through the nares elements to a patient's nostrils.

### CROSS REFERENCES TO RELATED APPLICATIONS

Not applicable.

### STATEMENT REGARDING FEDERALLY SPONSORED RESEARCH OR DEVELOPMENT

Not applicable.

### THE NAMES OR PARTIES TO A JOINT RESEARCH AGREEMENT

Not applicable.

### INCORPORATION BY REFERENCE OF MATERIAL SUBMITTED ON A COMPACT DISC

Not applicable.

### SEQUENCE LISTING

Not applicable.

### BACKGROUND OF THE INVENTION

### Field of the Invention

The present invention relates most generally to nebulizers and, more particularly, to a patient interface for a nebulizer system having multiple rapid injection ports for introducing medications into the nebulizer chamber during use, and still more particularly to a nebulizer system and nasal inhaler interface that delivers medications over extended periods of time through a patient's nose for absorption by nasal mucosa and lungs.

### Background Discussion

Intravenous delivery of medication is expensive and includes dangers and discomfort related to IV access. Medication delivered orally is less expensive, but gastric pH can destroy or alter medication, occasionally rendering it ineffective. Inhalation of medication can be limited by the toxic effects of the medication on the lungs. Nasal sprays make use of the nasal mucosa, but much of the medication is swallowed, thus limiting the absorption and the effectiveness during each spray given.

However, the controlled delivery of nasal medication over time can improve the effective absorption of each unit dose of a medication given while simultaneously reducing exposure to the lungs, as compared to such exposure when using oral inhalation devices.

It is well-known to use nebulizers to administer medications for respiratory conditions, such as asthma and chronic obstructive pulmonary disease. The inhaled aerosol is essentially delivered directly to the lungs. Nebulizers aerosolize or atomize liquid medications so that they can be entrained in air breathed in by the patient. Nebulizers aerosolize the medication using compressed air, mechanical means, or even ultrasound.

A typical interface for delivering medications to a patient using a nebulizer includes a face mask coupled to a breathing tube into which the aerosolized medication is dispersed. The patient generally takes in the medication orally.

Currently there is no device that allows for liquid medications in small and/or larger quantities to be administered over time through the nostrils so as to be directly absorbed through the nasal mucus membranes and lungs. Additionally, there are no nasal inhalation devices available that deliver medications over time via the nasal mucosa. With the potential advantages as yet unfulfilled, such a device is desirable and needed.

### BRIEF SUMMARY OF THE INVENTION

The inventive nasal inhaler for use with a nebulizer system as described herein has as its principal purpose the effective prolonged and continuous administration of medications through the nasal mucosa of a patient. The inhaler includes an interface tube configured for connection to a nebulizer chamber, an outlet tube, and a patient interface assembly. The inhaler interface includes a hollow interface coupler having a connector tube for coupling to the interface tube, a hollow removable concentrator connected to the interface coupler and having two concentrator outlet tubes, and two nares elements, one each removably connected to one of the concentrator outlet tubes. The interface coupler and the concentrator form a plenum through which medicated air under positive pressure from the nebulizer travels through the nares elements to a patient's nostrils.

The device provides for continuous nasal inhalation of medications. Such use enables more medication to be delivered and absorbed by the nasal mucosa than can be delivered using nasal inhalers. The device reduces exposure to the lung compared to orally inhaled products, and it prevents adverse gastric pH effects of the medication.

Importantly, the device reduces the amount of medication required by improving efficiency of delivery when given over time.

The nasal inhaler of the present invention can deliver all current nasal inhaled products, such as nasal steroids, antihistamines, saline rinses, and vasoconstrictors. Further, it allows better delivery of medications given through other routes currently as it is given over time, thus opening up the possibility of nasal administration of new or old medications given PO/IV/Inhaled/Tran-dermal.

The device further enables inhaled medications having beneficial effects for both nasal mucosa and lungs to be given together through one route rather than two (e.g., oral inhaled and nasal inhaled steroids).

Additionally, administration of medications through the nose over time may reduce lung toxicity and improve the delivery of medications such as inhaled insulin or antibiotics, targeting sinus infection, respiratory infection, and other infections. Inhalation of antitumor antibiotics directed at lung cancer using the inventive inhaler may be an alternative to IV administered medication. Antitrypsin delivery to nasal mucosa and lungs may also be an alternative administration route to IV administration. Pain medication and cannabis-based medicaments can also be administered using the inventive device.

A short and non-limiting list of medications that may be administered using the nasal inhaler of the present invention include: corticosteroids (various types); insulin; antibiotics (various types); chemotherapy drugs; pain medications; cannabis products/oils; nausea/vomiting medications; RA infusion medication; lupus IV medications; and stem cell therapy medications.

### BRIEF DESCRIPTION OF SEVERAL VIEWS OF THE DRAWINGS

The invention will be better understood and the objects and advantages of the invention, other than those set forth above, will become apparent when consideration is given to the following detailed description thereof. Such description makes reference to the annexed drawings wherein:

FIG. 1 is an upper perspective view of a nebulizer system incorporating the nasal inhaler of the present invention;

FIG. 2 is a top plan view showing the interface portion of the inhaler, featuring a Y-tube coupled to a side port for the rapid introduction of medications during long term use of the nasal inhaler;

FIG. 3 is the same view showing a main tube extending from the side port;

FIG. 4 is the same view showing the Y-tube coupled to side tubes;

FIG. 5 is the same view showing the side port capped with a plug;

FIG. 6 is a lower front perspective view of the inhaler interface;

FIG. 7 is a side view in elevation of the inventive system;

FIG. 8 is an upper front perspective view showing otherwise unseen operative structures in phantom;

FIG. 9 is an upper right partially exploded perspective view of the inhaler interface shown without side port inlet and tubing included; and

FIG. 10 is a fully exploded view thereof.

### DETAILED DESCRIPTION OF THE INVENTION

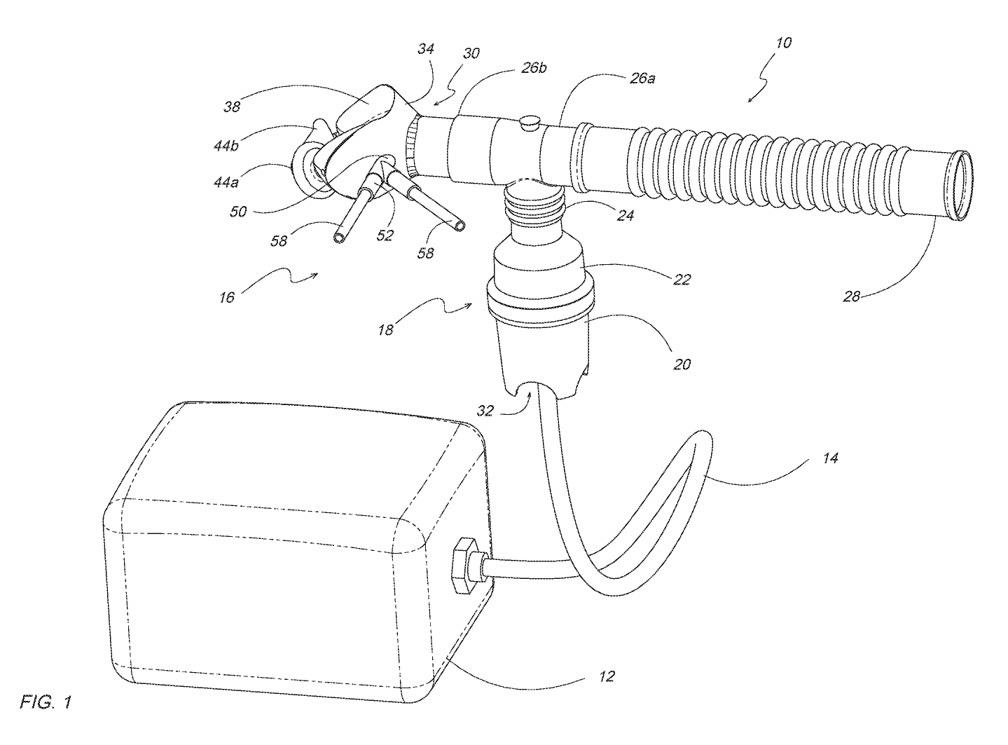

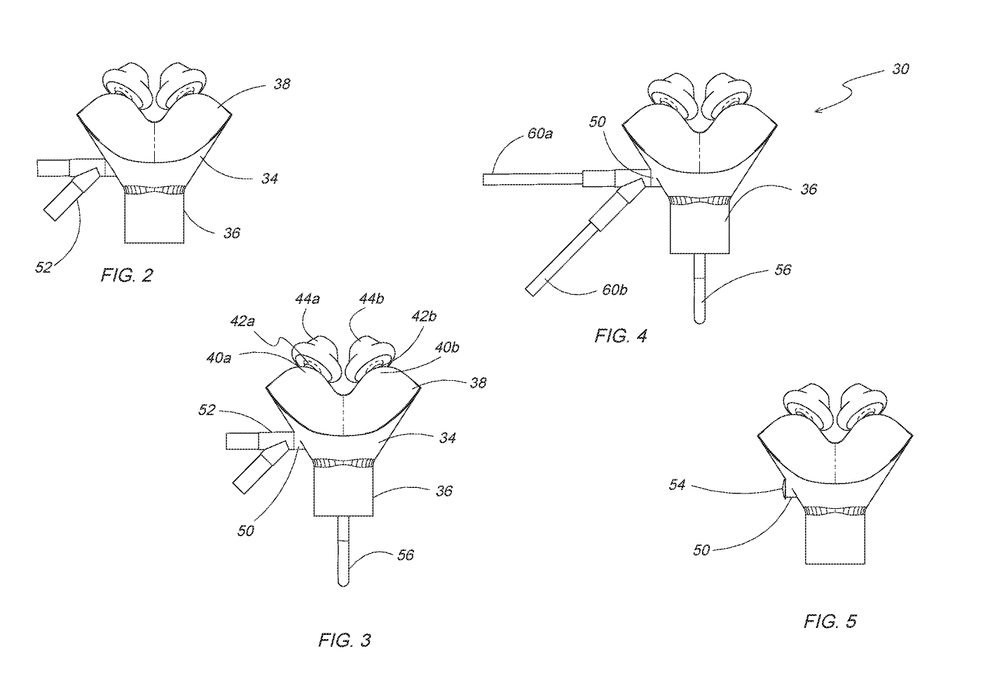

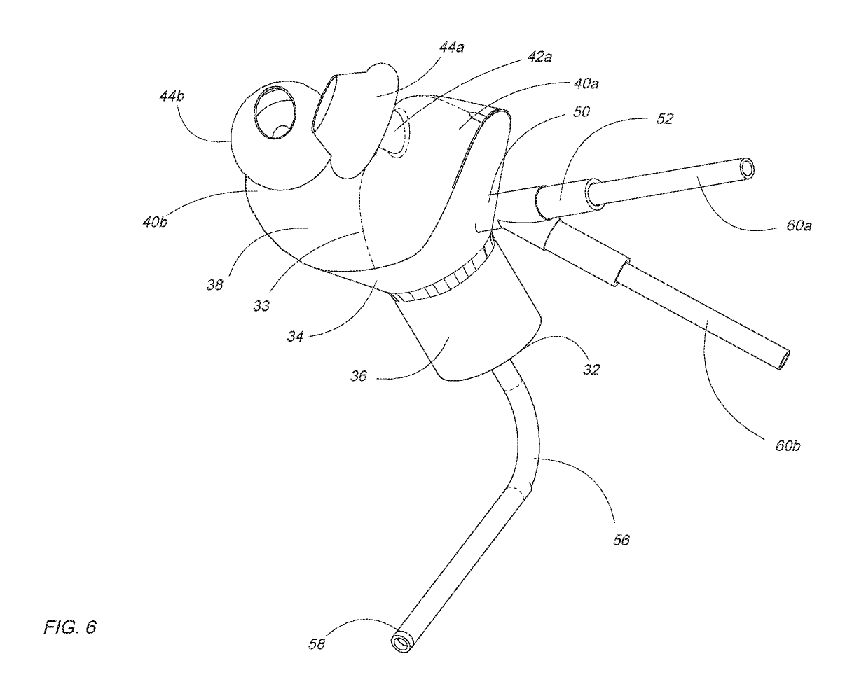

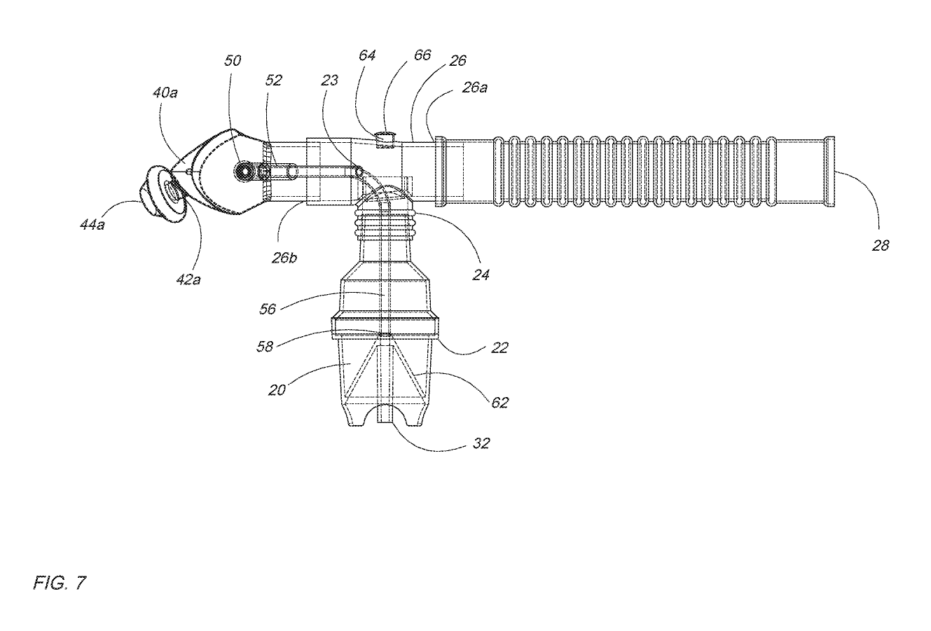

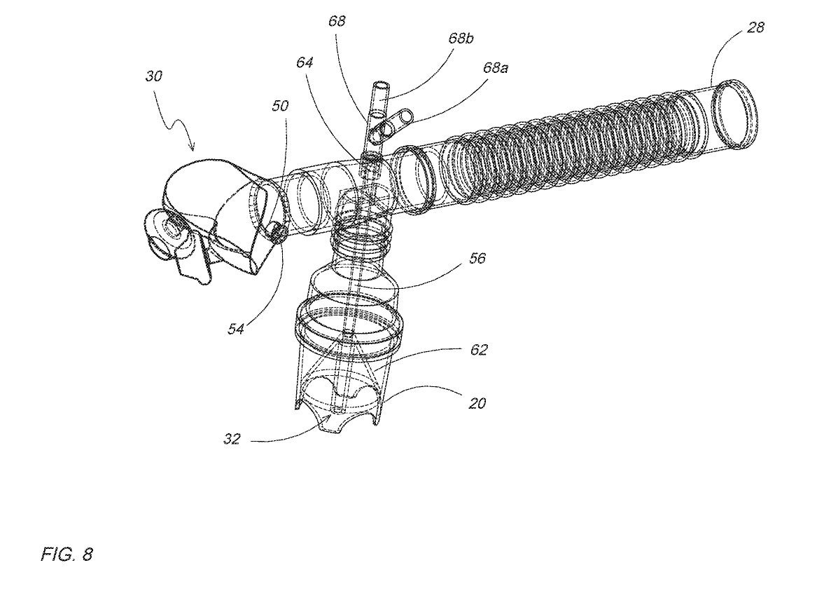

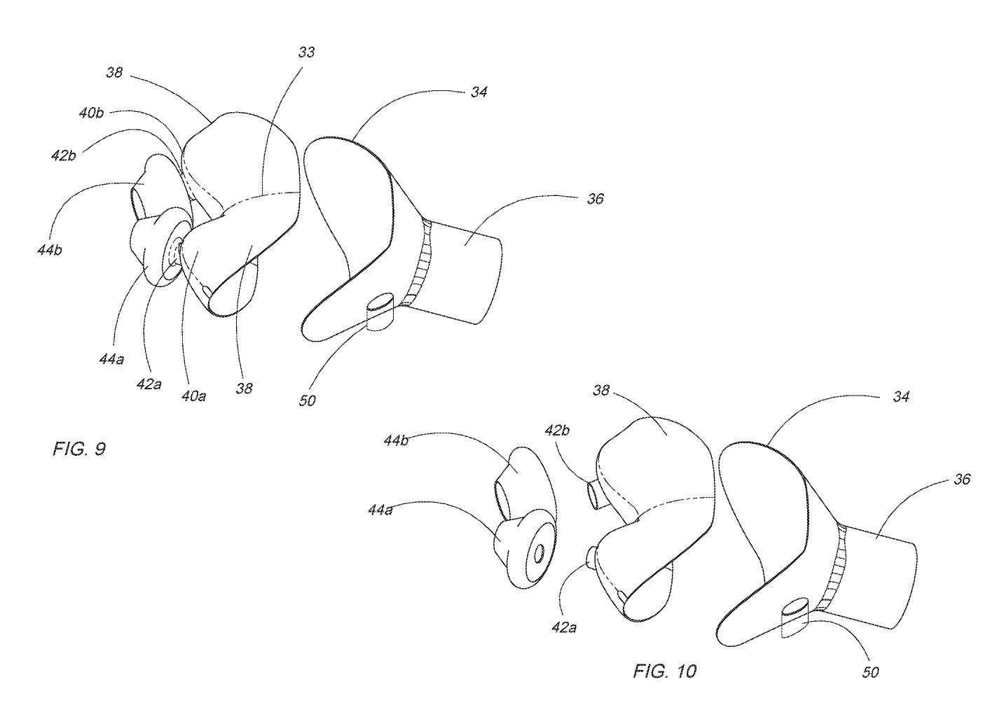

Referring to FIGS. 1 through 9, wherein like reference numerals refer to like components in the various views, there is illustrated therein a new and improved nasal inhaler for use with a nebulizer system, the inventive device denominated generally as 10 herein.

A system incorporating the inventive nasal inhaler includes a source of pressurized air 12 coupled by an air hose 14 to a dispenser assembly 16. The dispenser assembly includes a nebulizer chamber 18, which includes a base portion (or “medicine cup”) 20 covered by a cap 22, the latter open at the top and connected with a press-fit or threaded coupling 24 so as to be in fluid communication with a T-tube 26. The T-tube is, in turn, coupled at a first (outboard or exhalation) arm 26a to an air outlet (exhaust) tube 28 for patient exhalation to be released. On the second, opposite (inboard or inhalation) arm, 26b, the T-tube is connected to an inhaler interface 30 through which a patient inhales medicated air under pressure.

As in conventional nebulizer systems, the pressurized air source 12 (typically including a small air compressor) uses ambient air to generate pressurized air, which is then forced into and through an air hose 14 to the medicine cup 20 of the nebulizer chamber 18. The pressurized air hose 14 is connected to the nebulizer chamber at an air inlet 32. The pressurized air entering the nebulizer chamber entrains liquid medicine in the nebulizer chamber 18 and disperses it as a finely aerosolized mist. The droplets are driven by the moving air through an internal baffle 34, through the T-tube 26, and then through the inhaler interface to the patient. Unseen in the views are a first valve assembly interposed between the T-tube and the nebulizer chamber that prevent the backflow of air from patient exhalation into the nebulizer chamber 18 and a second valve assembly interposed between the nebulizer chamber and the exhaust tube that prevents air from being inhaled into the T-tube 26 through the exhaust tube 28.

The nasal inhaler includes a variety of inhaler interface elements and configurations for introducing medications through the inhaler interface or the T-tube. In embodiments the inhaler interface 30 includes a hollow interface coupler 34, which is a small plenum where air under positive pressure moves in both directions. The interface coupler includes an integral connector tube 36 which friction fits into the end of the inboard arm 26b of the T-tube 26.

The interface next includes a hollow removable concentrator 38, a hollow chamber symmetrically configured about a medial line 33, snap-fit connected to the interface coupler, where pressurized air containing entrained microspheres of aerosolized medicine are concentrated for delivery to the nostrils of a patient. The concentrator tapers into two rounded protuberant shoulders 40a, 40b, which focus the pressurized medication-containing air for output at two outlet tubes 42a, 42b. Nares elements 44a, 44b are pressed onto the outlet tubes and are inserted into a patient's nares. The nares elements are open both where they couple to the outlet tube and at their outer portion where they insert into patient nostrils, thereby placing the patient's nasal passages in fluid communication with the nebulizer chamber and the ambient atmosphere through the T-tube.

As will be appreciated, the inhaler interface device is a true nasal interface. This, as earlier noted, contrasts it with other nebulizer systems that employ full face masks for primary delivery of medicaments orally. The nares elements are spaced apart at a distance suitable for comfortable insertion into the nostrils of a vast majority of patient nares as determined by well-known anthropometric data. However, there is also a slight flexibility, both in the outlet tubes and in the removable concentrator to accommodate and address spacing in the nostrils of any particular patient. This is achieved by fabricating the concentrator and outlet tubes from a slightly resilient material, preferably a soft elastomeric polymer.

In embodiments, the inhaler interface includes one or more medication injection ports for the introduction of medications in addition to or to supplemental those already in the nebulizer chamber. A plurality of injection ports enables the administration of more of an initially introduced medication or complementary and/or synergistic medications simultaneously. These may be characterized as rapid injection ports, inasmuch as medications may be introduced into the nebulizer chamber in situ and without any need of any device disassembly or any removal of the inhaler from the patient.

Thus, in embodiments, the inhaler interface 30 may include a medication injection port 50 on a side of the inhaler concentrator 38. A connector 52, singular or Y-shaped, may be inserted into or coupled onto the injection port end. The injection port provides a connection point to place the nebulizer chamber 18 into fluid communication with sources of medication, the ambient atmosphere, or, selectively, to be capped and closed off with a plug 54. The injection port thus continues unbroken in an interior tube 56 that bends slightly inside the inlet port 50 in the inhaler concentrator 38, extends from the inhaler concentrator through the interface coupler tube 36 and inboard arm 26b of the T-tube 26, where it bends at a right angle downwardly and thereafter extends into the nebulizer chamber, where it terminates at an end 58 disposed above the chamber baffle 62 such that medications introduced through the injection port are, like those earlier introduced, aerosolized and sent back through the inhaler interface to the patient's nostrils.

In embodiments, the Y connector 52 may be extended to medication sources using side tubes 60a, 60b. In other embodiments, the first rapid injection port may be capped 54 or entirely eliminated, and a second rapid injection port 64 may be disposed in the T-tube 26 immediately above and in axial alignment with the cylindrical axis of the T-tube center (lower) arm 26c. When not in used, this port may be closed with a cap 66. When used for introducing medications, a connector, such as a Y-connector 68 with arms 68a, 68b, may be employed to provide multiple ports for the simultaneous or serial introduction of several medications.

From the foregoing, it will be evident that in its most essential aspect, the present invention is a nasal inhaler for use in a nebulizer system and includes an interface tube configured for connection to a nebulizer chamber, an outlet tube, and a patient interface assembly; an inhaler interface including a hollow interface coupler having a connector tube for coupling to the interface tube, and a structure which directs air and medicine to the patients nostrils (i.e., and airflow distribution and medication administration structure). The interface coupler and the concentrator form a plenum through which medicated air under positive pressure from the nebulizer travels through the airflow distribution and medication administration structure to a patient's nostrils; exhaled air passes through the plenum to and through the interface tube and out the outlet tube. In an embodiment, the airflow distribution and medication administration structure is a hollow removable concentrator and includes two concentrator outlet tubes and two nares elements, one each removably connected to one of the concentrator outlet tubes.

However, while the instant invention is a true nasal nebulizer, as is well known, aerosolized/nebulized medication can be administered to a patient's nostrils using other structures for distributing airflow and aerosolized medication. For instance, a facemask that covers the mouth and nose may be used. Disadvantageously, however, when using a facemask, medication may be deposited on the face and in and around the eyes, especially when used by a young child. Accordingly, using a structure for connecting the interface directly to a patient's nostrils is highly preferable, which is a principal purpose of the present invention. If a facemask is selected for airflow and medication distribution, patient understanding of use to maximize uptake in the nostrils is critical.

The above disclosure is sufficient to enable one of ordinary skill in the art to practice the invention, and provides various embodiments of the inventive concept as presently contemplated by the inventors. While there is provided herein a full and complete disclosure of the preferred embodiments of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like. For instance, it will be obvious to one having skill that the nebulizer may be brought into fluid communication with a patients nostrils through a variety of facemask configurations, including the concentrator described above, but also including convention facemasks and even facemask/mouthpiece combinations.

Therefore, the above description and illustrations should not be construed as limiting the scope of the invention, which is defined by the appended claims.

What is claimed as invention is:

1. A nasal inhaler for use in a nebulizer system, comprising:
an interface tube configured for connection to a nebulizer chamber, an outlet tube, and a patient interface assembly;
an inhaler interface including a hollow interface coupler having a connector tube for coupling to said interface tube;
an airflow distribution and medication administration structure in fluid communication with said inhaler interface to direct air flow from the nebulizer to a patient's nostrils;
wherein said interface coupler and said airflow distribution and medication administration structure form a plenum through which medicated air under positive pressure from the nebulizer travels through said airflow distribution and medication administration structure to a patient's nostrils and through which exhaled air under positive pressure moves to and through said interface tube and said outlet tube.

2. The nasal inhaler of claim 1, wherein said airflow distribution and medication administration structure comprises a removable hollow concentrator connected to said interface coupler and having two concentrator outlet tubes, and two nares elements, one each removably connected to one of said concentrator outlet tubes.

3. The nasal inhaler of claim 2, wherein medication air passes through said interface coupler and said nares elements to a patient's nostrils and through which exhaled air under positive pressure moves to and through said interface tube and said outlet tube.

4. The nasal inhaler of claim 2, wherein said connector tube is integrally formed in said interface coupler.

5. The nasal inhaler of claim 2, wherein said concentrator is symmetrically configured about a medial line.

6. The nasal inhaler of claim 2, wherein said concentrator connected to said interface coupler with a snap-fit connection.

7. The nasal inhaler of claim 2, wherein said concentrator includes two protuberant rounded shoulders for concentrating medicated air under positive pressure before the air passes through said concentrator outlet tubes.

8. The nasal inhaler of claim 2, further including at least one rapid injection port for the introduction of medication into the nebulizer chamber while the nebulizer system is in use.

9. The nasal inhaler of claim 2, wherein said at least one medication rapid injection port includes an inlet disposed on a side of said inhaler interface and a hollow tube in fluid communication with said inlet which passes through said inhaler interface and said interface tube, and terminates in the nebulizer chamber, wherein medication introduced through said inlet is deposited in the nebulizer chamber.

10. The nasal inhaler of claim 9, wherein said at least one medication rapid injection port includes an inlet disposed on said interface tube, and further includes a hollow tube in fluid communication with said inlet, said hollow tube passing through said interface tube and terminating in the nebulizer chamber, wherein medication introduced through said inlet is deposited in the nebulizer chamber.

11. The nasal inhaler of claim 9, wherein said at least one medication rapid injection port comprises:
a first rapid injection port having an inlet disposed on a side of said inhaler interface and a hollow tube in fluid communication with said inlet, said hollow tube passing through said inhaler interface, said interface tube, and terminating in the nebulizer chamber, wherein medication introduced through said first rapid injection port inlet is deposited in the nebulizer chamber, and
a second rapid injection port having a second rapid injection port inlet disposed on said interface tube and a hollow tube in fluid communication with said second rapid injection port inlet, said hollow tube passing through said interface tube and terminating in the nebulizer chamber, and a second , wherein medication introduced through said second rapid injection port inlet is deposited in the nebulizer chamber.

12. The nasal inhaler of claim 11, wherein each of said first and second rapid injection port may be selectively closed with a cap.

13. The nasal inhaler of claim 8, further including at least one branched connector enabling the simultaneous introduction of multiple medications through said at least one rapid injection port.

14. A nasal inhaler and nebulizer system for prolonged and continuous administration of medications, comprising:
a nebulizer interface configured to connect to a nebulizer chamber;
an outlet tube coupled to said nebulizer interface;
an inhaler interface connected to said nebulizer interface;
an airflow distribution and medication administration structure in fluid communication with said inhaler interface which brings the nebulizer into fluid communication with a patient's nostrils so as to direct air flow from the nebulizer through said inhaler interface and said airflow distribution and medication administration structure to the patient's nostrils.

15. The nasal inhaler and nebulizer system of claim 14, wherein said inhaler interface includes an interface coupler, and further wherein and interface coupler and said airflow distribution and medication administration structure form a volume through which medicated air under positive pressure from the nebulizer travels through said inhaler interface to a patient's nostrils on inhalation and through which exhaled air under positive pressure moves to and through said nebulizer interface to said outlet tube.

16. The nasal inhaler of claim 15, wherein said inhaler interface comprises a removable hollow concentrator connected to said interface coupler and further includes two concentrator outlet tubes and two nares elements, one each of said nares elements removably connected to one of said concentrator outlet tubes.

17. The nasal inhaler and nebulizer system of claim 16, wherein medication air passes through said interface coupler and said nares elements to a patient's nostrils and through which exhaled air moves to and through said interface tube and said concentrator outlet tubes.

18. The nasal inhaler and nebulizer system of claim 17, wherein said nares elements are protuberant rounded shoulders which focus and concentrate medicated air under pressure before the air passes through said concentrator outlet tubes to the patient's nostrils.

19. The nasal inhaler and nebulizer system of claim 14, further including at least one rapid injection port for the introduction of medication into the nebulizer chamber during use.

20. The nasal inhaler of claim 19, wherein said at least one medication rapid injection port includes an inlet disposed on a side of said inhaler interface and a tube in fluid communication with said inlet which passes through said inhaler interface and said interface tube, and terminates in the nebulizer chamber, wherein medication introduced through said inlet is deposited in the nebulizer chamber.
